(19) United States
(12) Patent Application Publication
(10) Pub. No.: US 2020/0008968 A1
(43) Pub. Date: Jan. 9, 2020

(54) Migraine Tension Headache Wrap

(71) Applicant: Cynthia Forster, Spring, TX (US)

(72) Inventor: Cynthia Forster, Spring, TX (US)

(21) Appl. No.: 16/506,432

(22) Filed: Jul. 9, 2019

Related U.S. Application Data
(60) Provisional application No. 62/695,435, filed on Jul. 9, 2018.

(51) Int. Cl. A61F 5/30; A61F 7/10; A61F 7/08

(52) U.S. Cl. CPC A61F 5/30; A61F 7/10; A61F 2007/0007; A61F 2007/108; A61F 7/08

(57) ABSTRACT

A migraine tension headache headband including a sleeve that is connected at both ends. The sleeve creates a closed annular body. The body is a headband. The sleeve has an elastic first material and a cushioned second material. The sleeve has at least one opening at a top portion of the sleeve. The opening has a hook and loop fastener located therein such that the opening can be secured in a closed position. The opening is sized to fit an ice pack or heat pack therein. The headband will be able to apply pressure to a majority of the wearers head and apply pressure thereby releasing the tension during a migraine.

### CROSS REFERENCE TO RELATED APPLICATIONS

This application claims the benefit of U.S. Provisional Application No. 62/695,435 filed on Jul. 9, 2018. The above identified patent application is herein incorporated by reference in its entirety to provide continuity of disclosure.

### BACKGROUND OF THE INVENTION

The present invention relates to migraine tension headache headbands. More particularly, the present invention provides a migraine tension headache headband wherein the same can be used to apply pressure to a user's head.

Many people suffer from migraine headaches. These headaches can be severely painful. Currently, there is nothing that will remove a migraine entirely. However, there are some tricks that will help to reduce the pain of the headache. Applying pressure can help to release tension and reduce the pain. It can be difficult to apply constant pressure around the entire head area. Without this uniform pressure the pain may just relocate to a different part of the head.

Consequently, there is a need in for an improvement in the art of migraine tension headache headbands. The present invention substantially diverges in design elements from the known art while at the same time solves a problem many people face when attempting to release tension in a person's head when having a migraine. In this regard the present invention substantially fulfills these needs.

### SUMMARY OF THE INVENTION

The present invention provides a migraine tension headache headband wherein the same can be utilized for providing convenience for the user when applying pressure to one's head in order to release migraine symptoms. The present invention includes a migraine tension headache headband comprising a sleeve, wherein the sleeve is connected together at each end. At least one opening in a top portion of the sleeve, wherein the opening goes from the top portion of the sleeve to a bottom portion of the sleeve.

Another object of the present invention is to provide a migraine tension headache headband further comprising a fastener located within the opening, wherein the opening can go from an open to a closed position.

Another object of the present invention is to provide a migraine tension headache headband wherein the sleeve is comprised of more than one material.

Another object of the present invention is to provide a migraine tension headache headband wherein an inner material is an elastic material.

Another object of the present invention is to provide a migraine tension headache headband wherein an outer material is a padding material.

Another object of the present invention is to provide a migraine tension headache headband wherein the sleeve is made from cotton.

Another object of the present invention is to provide a migraine tension headache headband wherein an ice pack is inserted into the sleeve.

Another object of the present invention is to provide a migraine tension headache headband wherein a heat pack is inserted into the sleeve.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the migraine tension headache headband.

FIG. 2 shows a cross-sectional view of an embodiment of the migraine tension headache headband.

FIG. 3 shows a perspective view of an embodiment of the migraine tension headache headband with an ice or heat pack located therein.

FIG. 4 shows a perspective in-use view of an embodiment of the migraine tension headache headband.

### DETAILED DESCRIPTION OF THE INVENTION

Reference is made herein to the attached drawings. Like reference numerals are used throughout the drawings to depict like or similar elements of the migraine tension headache headband. For the purposes of presenting a brief and clear description of the present invention, a preferred embodiment will be discussed as used for the migraine tension headache headband. The figures are intended for representative purposes only and should not be considered to be limiting in any respect.

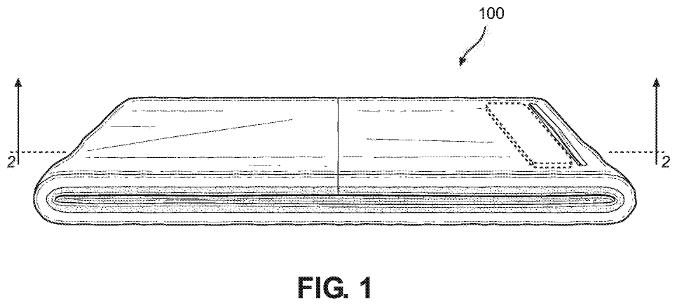

Referring now to FIG. 1, there is shown a perspective view of an embodiment of the migraine tension headache headband. The migraine tension headache headband comprises a sleeve. The sleeve is connected together at each end, such that a headband shape is formed. The migraine tension headache headband 100 is configured to fit around a wearers head. The migraine tension headache headband 100 is proportioned to fit over a wearer's ears and across the forehead.

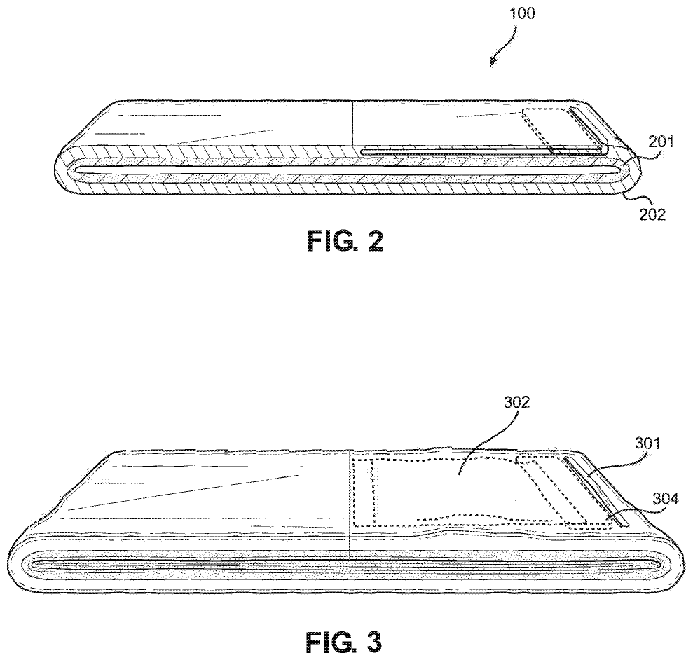

Referring now to FIG. 2, there is shown a cross-sectional view of an embodiment of the migraine tension headache headband. In one embodiment, the migraine tension headache headband 100 comprises multiple layers of material. In one embodiment the layers of material are comprised of a single material that is folded over to create the headband. In one embodiment the material is a cotton polyester blend. In one embodiment a first layer 201 is an elastic layer. The elastic layer 201 is a highly stretchable layer. This will ensure that the migraine tension headache headband 100 will apply pressure to the wearer. The first layer 201 will flex out to be placed over the wearer's head then tighten down applying pressure to the wearer.

The migraine tension headache headband 100 has a second layer 202. In one embodiment the second layer is comprised of the same material at the first layer 201 and results from a fold in the material. The second layer 202 is a cushion layer. In one embodiment the cushion layer 202 is a padded material. The cushion layer will allow for the wearer to experience comfort when the migraine tension headache headband 100 is worn. In one embodiment, the second layer 202 also provides warmth to the wearer.

Referring now to FIG. 3, there is shown a perspective view of an embodiment of the migraine tension headache headband with an ice or heat pack located therein. In one embodiment, the migraine tension headache headband has an opening 301 in an external side of the migraine tension headache headband 100. In one embodiment, there is an opening on two sides of the migraine tension headache headband 100. This opening 301 will lead to an internal compartment 302. In another embodiment, both openings will lead to individual internal compartments. In one embodiment one of the compartments is located acrossed forehead and one of the compartments is located on the base of the skull. In another embodiment, the openings will lead to a single connected internal compartment. In one embodiment the internal compartment 302 is located between the first and second layer. In another embodiment, the internal compartment located on the inside of the migraine tension headache headband 100.

The internal compartment is configured to fit a heat pack or ice pack 303. This will allow for a wearer to apply heat or ice to their head. In one embodiment, there will be a closure device 304 located within the compartment. The closure device will be located just within the opening. In one embodiment the closure device goes all the way across the opening. In another embodiment the closure device is located in the middle of the opening. This will allow a user to seal the compartment to securely hold items within the internal compartment. In one embodiment, the closure device 304 is a hook and loop fastener.

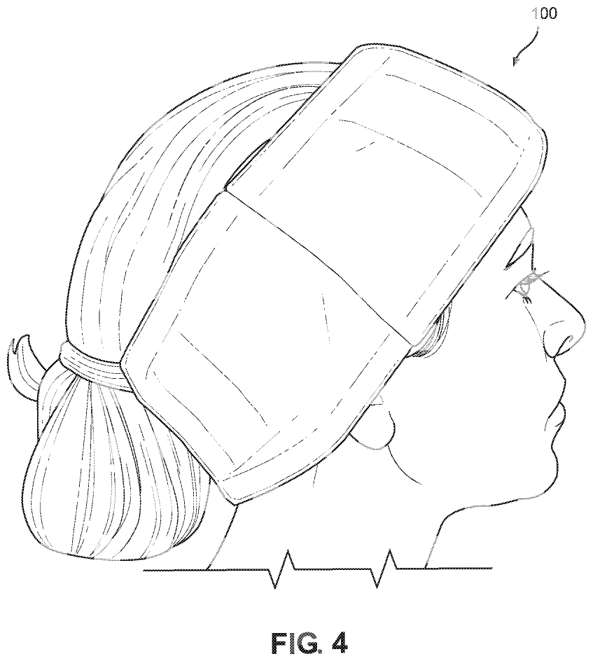

Referring now to FIG. 4, there is shown a perspective in-use view of an embodiment of the migraine tension headache headband. In use, the migraine tension headache headband 100 covers the wearer's forehead and the sides of the head. In one embodiment the migraine tension headache headband 100 covers the wearer's ears. The top of the migraine tension headache headband is opened to allow the top of the wearer's head to come through. This will allow a user to pull the head band lower or position it higher to achieve maximum comfort.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) A migraine tension headache headband, comprising:
a sleeve, wherein the sleeve is connected together at each end;
at least one opening in a top portion of the sleeve, wherein the opening goes from the top portion of the sleeve to a bottom portion of the sleeve.

2) The migraine tension headache headband of claim 1, further comprising a fastener located within the opening, wherein the opening can go from an open to a closed position.

3) The migraine tension headache headband of claim 1, wherein the sleeve is comprised of more than one material.

4) The migraine tension headache headband of claim 3, wherein an inner material is an elastic material.

5) The migraine tension headache headband of claim 3, wherein an outer material is a padding material.

6) The migraine tension headache headband of claim 1, wherein an ice pack is inserted into the sleeve.

7) The migraine tension headache headband of claim 1, wherein a heat pack is inserted into the sleeve.

8) A migraine tension headache headband, comprising:
a sleeve, wherein the sleeve is connected together at each end;
the sleeve is comprised of more than one material, wherein an inner material is an elastic material and wherein an outer material is a padding material
at least one opening in a top portion of the sleeve, wherein the opening goes from the top portion of the sleeve to a bottom portion of the sleeve.

9) The migraine tension headache headband of claim 8, wherein an ice pack is inserted into the sleeve.

10) The migraine tension headache headband of claim 8, wherein a heat pack is inserted into the sleeve.

11) The migraine tension headache headband of claim 8, further comprising a fastener located within the opening, wherein the opening can go from an open to a closed position.
